(19) United States
(12) Patent Application Publication
(10) Pub. No.: US 2022/0076837 A1
(43) Pub. Date: Mar. 10, 2022

(54) HEALTH EVALUATION SYSTEM, HEALTH EVALUATION SERVER AND HEALTH EVALUATION PROGRAM

(71) Applicant: FINC TECHNOLOGIES INC., Tokyo (JP)

(72) Inventors: Yosuke SUZUKI, Tokyo (JP); Akiko MIYAZAKI, Tokyo (JP); Yuji MIZOGUCHI, Tokyo (JP)

(21) Appl. No.: 17/275,607

(22) PCT Filed: Aug. 26, 2019
(86) PCT No.: PCT/JP2019/033246

§ 371 Date: Mar. 11, 2021

(30) Foreign Application Priority Data
Sep. 11, 2018 (JP) 2018-169526

(51) Int. Cl. G16H 50/30; G16H 10/20

(52) U.S. Cl. CPC G16H 50/30; G16H 10/20

(57) ABSTRACT

A technique for suitably performing a health evaluation of individuals and organizations. A question information database of a management server stores a physical question group which has multiple question items based on the viewpoint of current physical states and lifestyle habits, a mental question group which has multiple question items based on the viewpoint of psychological state, and an engagement question group which has multiple question items based on the viewpoint of the user's motivation. The management server sends thea user terminal question information generated on the basis of the physical question group, the mental question group and the engagement question group. The user terminal sends the management server the user's responses to the received question information. The management server generates advice information and a total score based on the responses and provides these to the user.

### TECHNICAL FIELD

The present invention relates to a health evaluation system, a health evaluation server and a heath evaluation program.

### BACKGROUND ART

In recent years, health guidance services that receive expert guidance have become widespread in line with the rise in health awareness such as dieting and prevention of metabolic syndrome. In such guidance, the efficiency of guidance is improved by forcing a guidance target person (hereinafter referred to as “user”) to answer a questionnaire (sometimes called survey, interview, etc.) regarding lifestyle habits or the like in advance.

On the other hand, there are also questionnaires that are obliged to answer the number of items prescribed by a law, such as so-called stress checks.

### PRIOR ART DOCUMENTS

### Patent Literature

Patent Literature 1: Japanese Unexamined Patent Publication No. 2017-21660

### SUMMARY OF THE INVENTION

### Technical Problem

However, for the purpose of such questionnaires, there are many cases where only question items related to specific areas such as lifestyle habits and satisfaction are given, and the user's health condition cannot be evaluated in consideration of multifaceted evaluation and analysis and multiple factors.

In addition, such an evaluation is an individual evaluation, and in order to evaluate and analyze the tendency of the entire organization and the problems associated with the organization, it is necessary to approach it in a different manner.

Therefore, the present invention aims to provide a technique that can properly perform the health evaluation of individuals and organizations.

### Technical Solution

According to the present invention, there is provided a method for supporting a user's answering operation to a question by using a user terminal having a display unit, the method comprising the steps of: displaying a first question item in a first predetermined area of the display unit, and displaying a second question item in a second predetermined area, accepting an answering operation from the user for the first question item, moving the first question item out of the first predetermined area along a predetermined direction, and moving the second question item from the second predetermined area to the first predetermined area along the predetermined direction, and receiving an answering operation from the user for the second question item.

### Advantageous Effects

According to the present invention, a technique for appropriately performing a health evaluation of individuals and/or organizations can be provided.

### BRIEF DESCRIPTION OF THE DRAWING

FIG. 1 is a view conceptually showing the structure of a question according to an embodiment of the present invention.

FIG. 2 is a view showing a configuration example of a questionnaire system according to the present invention.

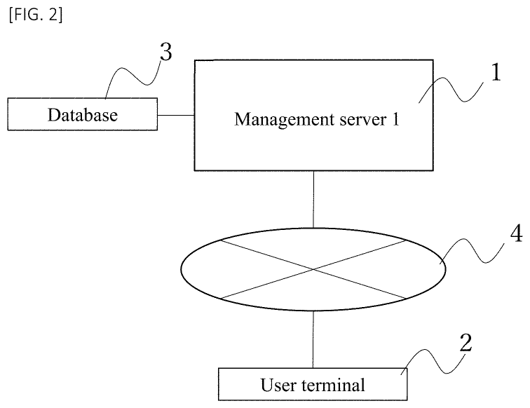

FIG. 3 is a block diagram showing a configuration of a management server shown in FIG. 2.

FIG. 4 is a diagram showing the concept of input and output according to the present embodiment.

FIG. 5 is a diagram showing an object analysed by the system according to the present embodiment.

FIG. 6 is an example of a physical question according to the present embodiment.

FIG. 7 is an example of a mental question according to the present embodiment.

FIG. 8 is an example of an engagement question according to the present embodiment.

FIG. 9 is a processing diagram of a system according to the present embodiment.

FIG. 10 is an example of a screen display of a user terminal.

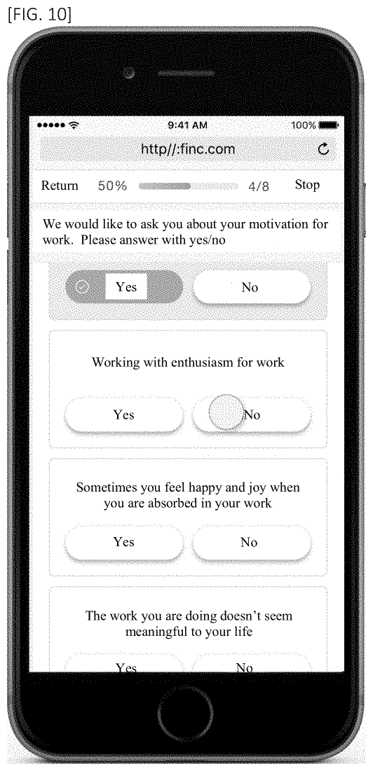

FIG. 11 is an example of a screen display of a user terminal.

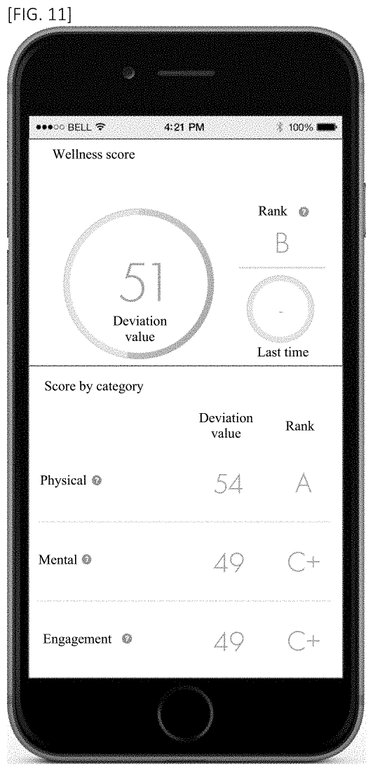

### DETAILED DESCRIPTION OF THE EMBODIMENTS

Hereinafter, one embodiment of the present invention will be described with reference to the accompanying drawings.

[Item 1]

A health evaluation system which is provided with a user terminal and a management server having a question information database,

wherein the question information database stores a physical question group which has multiple question items based on the viewpoint of current physical conditions and lifestyle habits, a mental question group which has multiple question items based on the viewpoint of psychological state, and an engagement question group which has multiple question items based on the viewpoint of the user's motivation,

wherein the management server transmits question information generated on the basis of the physical question group, the mental question group, and the engagement question group to the user terminal,

wherein the user terminal transmits the user's answer to the received question information to the management server, and

wherein the management server generates total score and advice information based on the answer and provides these to the user.

[Item 2]

The health evaluation system as set forth in Item 1,

wherein the management server calculates the physical score, the mental score, and the engagement score, respectively, based on the answers to respective question items of the physical question group, the mental question group, and the engagement question group, and

calculates the total score based on the physical score, the mental score, and the engagement score.

[Item 3]

The health evaluation system as set forth in Item 2,

wherein the management server classifies the physical score, the mental score, the total score, and the total score into a predetermined hierarchy, and

provides the classified physical score, mental score, total score, and information of the total score to the user terminal.

[Item 4]

The health evaluation system as set forth in Item 2 or 3,

wherein the management server calculates the physical score, the mental score and the engagement score, and the total score of all users belonging to a predetermined organization, and transmits the result to the organization management terminal that manages the organization.

[Item 5]

The health evaluation system as set forth in any one of Items 1 to 4,

wherein the management server permits a predetermined administrator to edit the contents of the question items included in the physical question group, the mental question group, and the engagement question group according to the user. [Item 6]

A health evaluation server having a question information database,

wherein the question information database stores a physical question group which has multiple question items based on the viewpoint of current physical conditions and lifestyle habits, a mental question group which has multiple question items based on the viewpoint of psychological state, and an engagement question group which has multiple question items based on the viewpoint of the user's assessment of the environment in which the user is placed, and

wherein the health evaluation server comprises:

a means for transmitting question information generated on the basis of the physical question group, the mental question group, and the engagement question group to a user terminal,

a means for receiving the user's answer to the question information from the user terminal, and

a means by which the management server generates total score and advice information based on the answer and provides these to the user.

[Item 7]

A health evaluation program,

wherein a health evaluation terminal has a question information database which stores a physical question group having multiple question items based on the viewpoint of current physical conditions and lifestyle habits, a mental question group having multiple question items based on the viewpoint of psychological state, and an engagement question group having multiple question items based on the viewpoint of evaluation of the user's environment, and

wherein the health evaluation program enables the health evaluation terminal to function as:

a means for transmitting question information generated based on the physical question group, the mental question group, and the engagement question group to the user terminal,

a means for receiving the user's answer to the question information from the user terminal, and

a means by which the management server generates total score and advice information based on the answer and provides these to the user.

### SUMMARY

The present invention is a health evaluation system (hereafter, simply referred to as “system”) which comprehensively visualizes a scale indicating “what kind of reaction is occurring” and “what is a factor” with respect to the effect achieved through the improvement of the conditions of organizations and individuals. More specifically, it relates to a technique which quantitatively evaluates and analyses the health condition of an individual and the health condition of the entire corporate organization to which multiple individuals belong and visualizes the result.

This system allows a user to answer to multiple question items (physical questions) based on the viewpoint of current physical conditions and lifestyle habits, multiple question items (mental questions) based on the viewpoint of psychological state, and multiple question items (engagement questions) based on the viewpoint of user motivation, calculates the physical score, mental score, and engagement score based on the answer results for each question item, and further calculates the wellness score based on these scores.

### <Configuration>

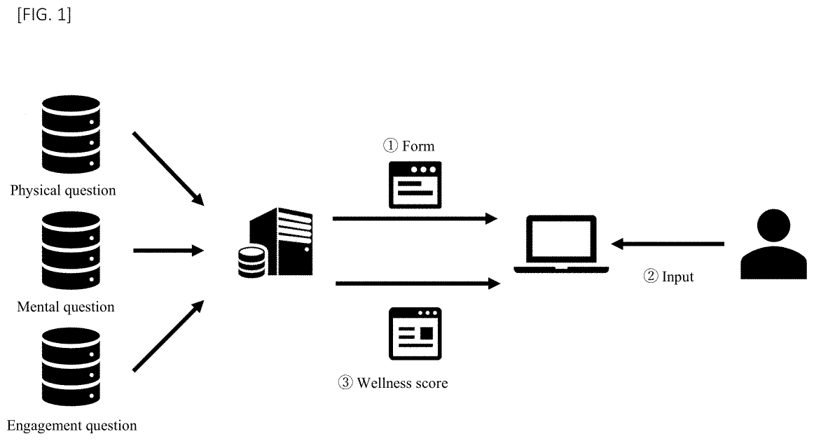

As shown in FIG. 1, this system includes a management server 1 and a user terminal 2. The management server 1 is connected to a database (described later). The management server 1 and the user terminal 2 are communicably connected to each other via a network 4. Further, this configuration is merely one example, and a plurality of elements may be integrated to form one function/configuration, including the description below, or other configurations may be included.

### <Management Server>

As shown in the figure, the management server 1 is connected to a database 3 and constitutes a part of the system. The management server 1 may be a general-purpose computer such as a workstation or a personal computer, or may be logically realized by cloud computing.

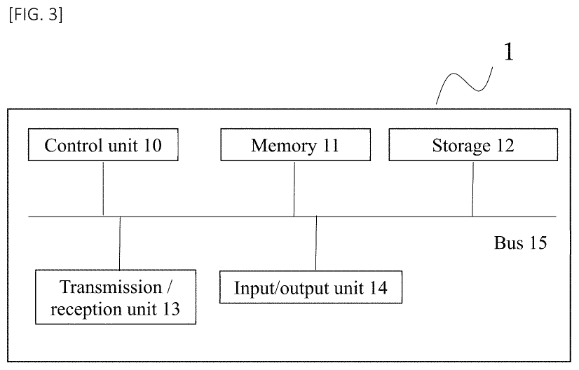

As shown in FIG. 3, the management server 1 includes at least a control unit 10, a memory 11, a storage 12, a transmission/reception unit 13, an input/output unit 14, and the like, and these are electrically connected to each other through a bus 15.

The control unit 10 is an arithmetic device that controls the operation of the entire management server 1, controls the transmission and reception of data between each element, and performs information processing necessary for application execution and authentication processing. For example, the control unit 10 is a CPU (Central Processing Unit), and executes each information processing by executing a program or the like stored in the storage 12 and deployed in the memory 11.

The memory 11 includes a main memory composed of a volatile storage device such as a DRAM (Dynamic Random Access Memory) and an auxiliary memory composed of a non-volatile storage device such as a flash memory or an HDD (Hard Disk Drive). The memory 11 is used as a work area or the like of the processor 10, and also stores a BIOS (Basic Input/Output System) executed when the management server 1 is started, various setting information, and the like.

The storage 12 stores various programs such as application programs. A database (not shown) storing data used for each processing may be built in the storage 12.

The transmission/reception unit 13 connects the management server 1 to the network. The transmission/reception unit 13 may be provided with a short-range communication interface such as Bluetooth (registered trademark) or Wi-Fi (registered trademark).

The input/output unit 14 is an information input device such as a keyboard and a mouse, and an output device such as a display.

The bus 15 is commonly connected to each of the above elements and transmits, for example, an address signal, a data signal, and various control signals.

### <Data>

Returning to FIG. 1, the outline of the operation flow of the present embodiment is described, and then the data used in this system is described.

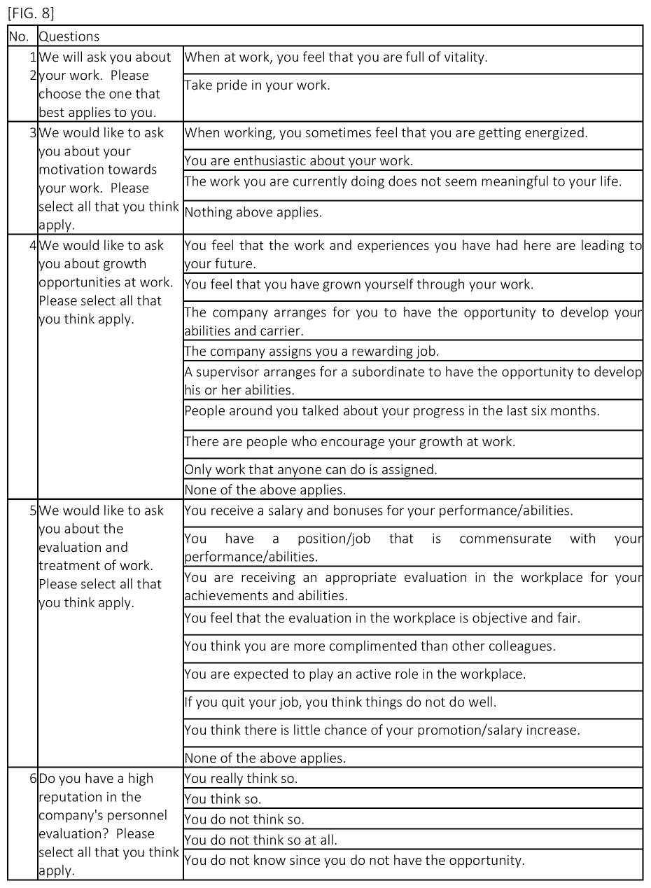

As shown in the figure, the management server 1 is connected to a database that stores a physical question, a mental question and an engagement question, respectively. The management server 1 transmits a predetermined survey (questionnaire) form to a user terminal. The survey form is displayed on the user terminal such as a smartphone and prompts the user to respond, for example, as shown in FIG. 8.

As shown in FIG. 8, the survey form can adopt various formats, such as a format which answers to the question in accordance with the option “yes” or “no”, or a format that selects the answer from a plurality of options. When the user answers all the questions, the answer information is recorded (temporarily or continuously) in the user terminal and transmitted to a management server 1.

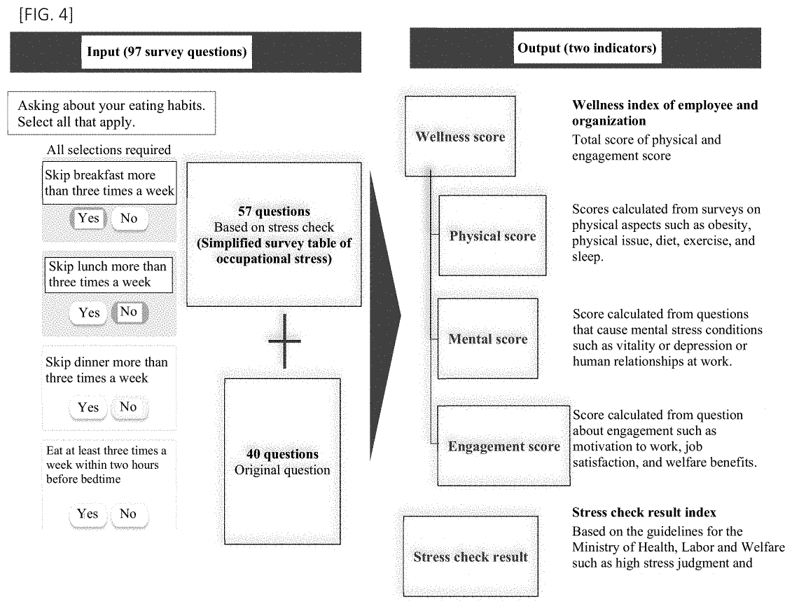

As shown in FIG. 4, when considering the management server 1 according to the present embodiment from the viewpoint of “input” and “output”, what can be grasped as input to the management server 1 is “answer information from the user”, and what can be grasped as the output from the management server 1 is the “score based on the answer information”. Further. in the description so far, the word “score” is used, but terms such as score, index, point, deviation value, rank, etc. can be adopted as appropriate.

More specifically, as shown in the figure, as an input, answer information of 97 questions in total, including 57 questions stipulated in the Simple Occupational Stress Questionnaire based on the so-called stress check, and 40 supplementary questions for questions that cannot be covered by the stress check, is provided from the user. Each of the 97 questions is categorized by a physical question, a mental question or an engagement question (alone/allowing duplication). The management server 1 aggregates the answer information for each of the three categories based on the results of the answers to the 97 questions, and calculates them as a physical score, a mental score, and an engagement score, respectively. In addition, the wellness score, which is the sum of these scores, is also calculated at the same time.

The “physical question-physical score” according to the present embodiment includes a viewpoint of the user's current physical conditions and lifestyle habits, the “mental question-mental score” includes the viewpoint of the user's psychological state, and the “engagement question-engagement score” includes the viewpoint of the user's motivation. Further, the wellness score is the total score of the physical score, the mental score and the engagement score. Further, the wellness score is not a mere sum of these, but may be appropriately adjusted by multiplying by a predetermined coefficient or by applying a slope.

As a specific example, the physical score is calculated from physical questions such as obesity, physical issues, diet, exercise, and sleep. The mental score according to the present embodiment is, for example, calculated from questions that cause a state of mental stress such as vitality and depression, workplace relationships, and the like. The engagement score according to the present embodiment is, for example, calculated from questions related to engagement such as motivation to work, job satisfaction, and welfare. The wellness score calculated from these is used as an index of the wellness of employees and organizations.

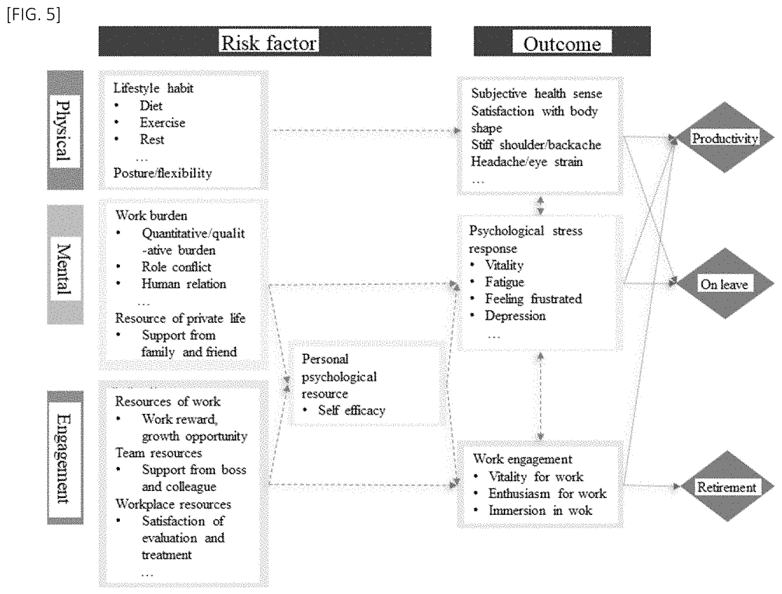

Referring to FIG. 5, the outcomes that can be analyzed from the physical question, the mental question, and the engagement question are described. As shown in the figure, the risk factors analyzed by physical questions relate to, for example, lifestyle habits (diet, exercise, rest, etc.), posture, flexibility, and the like. The risk factors analyzed by mental questions are, for example, work burdens (quantitative burden, qualitative burden, role conflict, human relation, etc.) and personal life resources (support from family and friends). The risk factors analyzed by engagement questions are related to, for example, work resources (work worthiness, growth opportunities), team resources (boss/colleague support), workplace resources (evaluation/treatment satisfaction), and the like.

As shown in FIG. 5, in the present embodiment, further, information obtained from information sources other than the survey of information that evaluates productivity (performance data, progress data, etc.), or information on whether or not to be on a leave from work (personnel data, attendance data, etc.), information on retirement plans and facts (personnel data, etc.), and the results of the above-described survey may be analyzed in a combined manner.

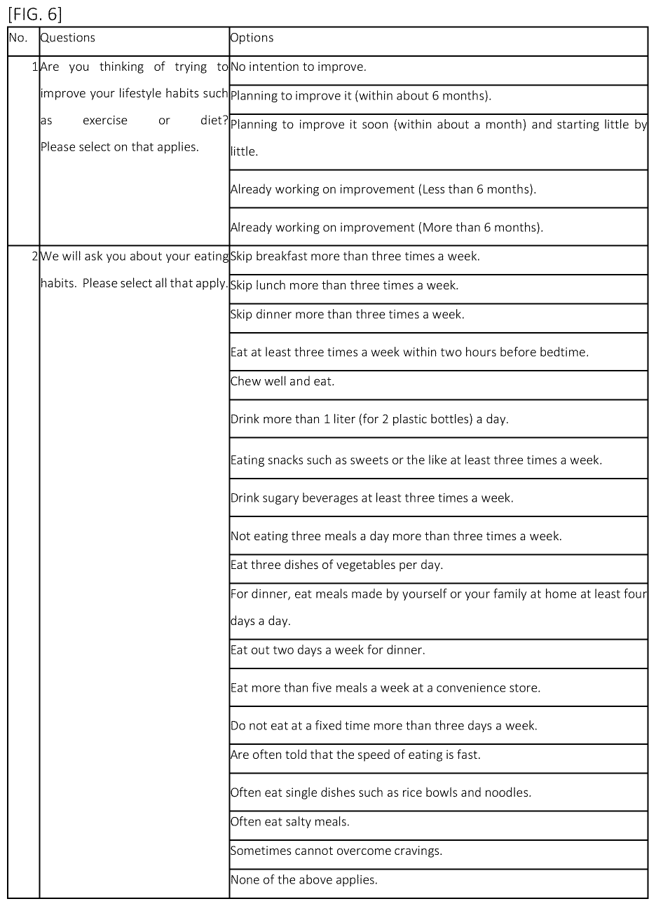

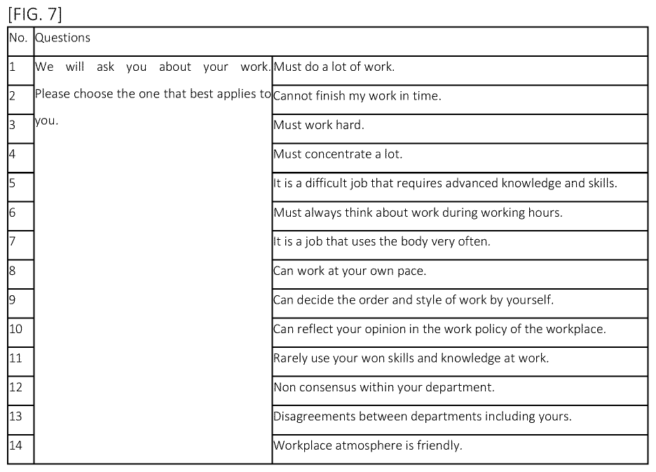

Further, FIGS. 6 to 8 show examples of physical question items, mental question items, and engagement question items, respectively.

### <Operation>

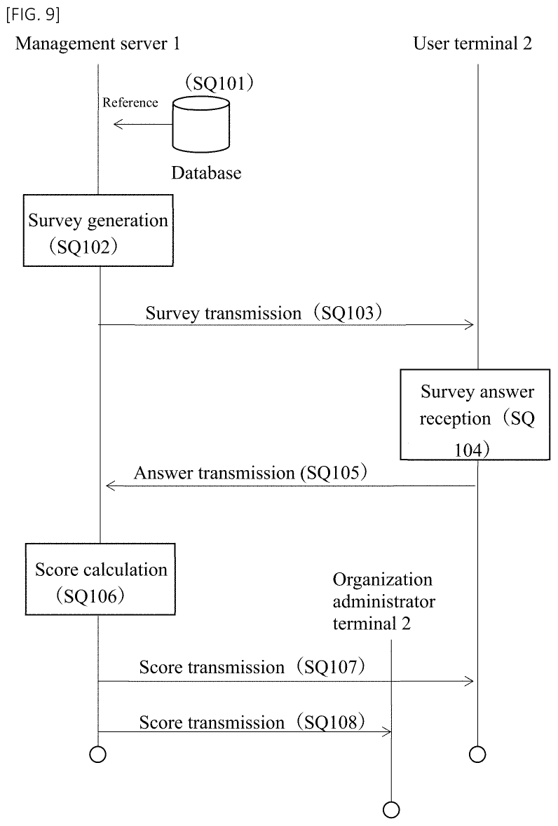

Next, as shown in FIG. 9, the operation of the system according to the present embodiment will be described. Further, the following operation example is one example, and the processing timing, target, and contents can be changed as appropriate.

As shown in the figure, the management server 1 refers to a database in which the above-mentioned questions (physical question, mental question and engagement question) are stored (SQ101), and generates a survey for transmission to the user terminal (SQ102). The generated survey is transmitted from the management tool 1 to the user terminal 2 (SQ103). In the user terminal 2, the user is urged to answer the survey (SQ104). The answered contents are transmitted to the management server 1 (SQ105). Further, regarding the survey answers, the user may be urged to answer at once, or may be partially answered little by little.

The management server 1 calculates each score based on the answer result (SQ106). For the calculation, for example, the following ideas are adopted, but these are merely examples.

$$f = {\sum }_{k = 1}^{n} p \{ {α}_{1} {Qf}_{1} + {α}_{2} {Qf}_{2} + … + {α}_{n} {Qk}_{n} \} \qquad [ Formula 1 ]$$

wherein, the symbols in the formula have the following meanings:

f: Physical score,

Qf₁, Qf₂, . . . Qfₙ: score assigned to the options answered by the user in the physical question,

α₁, α₂, . . . αₙ: Coefficients considered for each question, and

p: Coefficient considered for the entire physical score.

$$m = {\sum }_{k = 1}^{n} q \{ {β}_{1} Q {m}_{1} + {β}_{2} Q {m}_{2} + … + {β}_{n} Q {m}_{n} \} \qquad [ Formula 2 ]$$

wherein, the symbols in the formula have the following meanings:

m: Mental score,

Qm₁, Qm₂, . . . Qmₙ: score assigned to the options answered by the user in the mental question,

β₁, β₂, . . . βₙ: Coefficients considered for each question, and

q: Coefficient considered for the entire mental score.

$$e = {\sum }_{k = 1}^{n} r \{ {γ}_{1} {Qe}_{1} + {γ}_{2} {Qe}_{2} + … + {γ}_{n} {Qe}_{n} \} \qquad [ Formula 3 ]$$

wherein, the symbols in the formula have the following meanings.

e: Engagement score

Qe₁, Qe₂, . . . Qeₙ: Scores assigned to the options answered by the user in the engagement question,

γ₁, γ₂, . . . γₙ: Coefficients considered for each question, and

r: Coefficient considered for the entire engagement score.

W=s{f+m+e} [Formula 4]

The wellness score W can be represented by the sum of the above-mentioned physical score f, mental score m, and engagement score. Further, a predetermined coefficient s may be multiplied for each company, each organization, or each individual, if necessary.

The above calculation formula is merely one example. In addition to the above, for example, an element to be scored as a relative evaluation for the whole population may be added. For example, it can be evaluated as a deviation value with respect to the distribution of the overall score. In addition, if necessary, it may be determined which rank (position) one's organization is in a population such as a specific industry, service, or region. According to such a configuration, it is possible to perform quantitative evaluation in consideration of the actual situation (reality).

Continuing to refer to FIG. 9, the management server 1 transmits the calculated wellness score to the user terminal 2 (SQ107). Thereby, the user can see the result.

The system according to this embodiment discloses, for example, a series of scores such as a wellness score to the personnel manager and the manager of the company. Thereby, it becomes possible to objectively grasp the health condition of employees of the organization.

The above-mentioned scores (wellness score, physical score, mental score, engagement score) may be displayed as numerical values. However, for example, the superiority or inferiority (high or low) of the score may be displayed by another comparison method, such as a three-stage evaluation of A to C.

Further, for example, the content of each question and the coefficient assigned to the answer to each question may be appropriately changed for each user and each organization. Thereby, the content of the survey can be customized according to various factors such as industry and business type, and it is possible to conduct a more realistic survey.

### DESCRIPTION OF REFERENCE NUMERALS

1: Health management server

2: User terminal

3: Database

4: Network

1. A health evaluation system which is provided with a management server having a question information database,
wherein the question information database stores a physical question group which has multiple question items based on the viewpoint of current physical conditions and lifestyle habits, a mental question group which has multiple question items based on the viewpoint of psychological state, and an engagement question group which has multiple question items based on the viewpoint of the user's motivation,
wherein the management server transmits question information generated on the basis of the physical question group, the mental question group, and the engagement question group to a user terminal,
wherein the user terminal transmits the user's answer to the received question information to the management server, and
wherein the management server generates total score and advice information based on the answer and provides these to the user.

2. The health evaluation system according to claim 1,
wherein the management server calculates the physical score, the mental score, and the engagement score, respectively, based on the answers to respective question items of the physical question group, the mental question group, and the engagement question group, and
calculates the total score based on the physical score, the mental score, and the engagement score.

3. The health evaluation system according to claim 2,
wherein the management server classifies the physical score, the mental score, the total score, and the total score into a predetermined hierarchy, and
provides the classified physical score, mental score, total score, and infoiniation of the total score to the user terminal.

4. The health evaluation system according to claim 2,
wherein the management server calculates the physical score, the mental score and the engagement score, and the total score of all users belonging to a predetermined organization, and transmits the result to a organization management terminal that manages the organization.

5. The health evaluation system according to claim 1,
wherein the management server enables a predetermined administrator to edit the contents of the question items included in the physical question group, the mental question group, and the engagement question group according to the user.

6. A health evaluation server having a question information database,
wherein the question information database stores a physical question group which has multiple question items based on the viewpoint of current physical conditions and lifestyle habits, a mental question group which has multiple question items based on the viewpoint of psychological state, and an engagement question group which has multiple question items based on the viewpoint of the user's motivation, and
wherein the health evaluation server comprises:
a means for transmitting question information generated on the basis of the physical question group, the mental question group, and the engagement question group to a user terminal,
a means for receiving the user's answer to the question information from the user terminal, and
a means by which the management server generates total score and advice information based on the answer and provides these to the user.

7. A health evaluation program,
wherein a health evaluation terminal has a question information database which stores a physical question group having multiple question items based on the viewpoint of current physical conditions and lifestyle habits, a mental question group having multiple question items based on the viewpoint of psychological state, and an engagement question group having multiple question items based on the viewpoint of evaluation of the user's environment, and
wherein the health evaluation program enables the health evaluation terminal to function as:
a means for transmitting question information generated based on the physical question group;
the mental question group, and the engagement question group to the user terminal;
a means for receiving the user's answer to the question infoiniation from the user terminal; and
a means by which the management server generates total score and advice information based on the answer and provides these to the user.

8. The health evaluation system according to claim 3,
wherein the management server classifies the physical score, the mental score, the total score, and the total score into a predetermined hierarchy, and
provides the classified physical score, mental score, total score, and information of the total score to the user terminal.

9. The health evaluation system according to claim 2,
wherein the management server enables a predetermined administrator to edit the contents of the question items included in the physical question group, the mental question group, and the engagement question group according to the user.

10. The health evaluation system according to claim 3,
wherein the management server enables a predetermined administrator to edit the contents of the question items included in the physical question group, the mental question group, and the engagement question group according to the user.

11. The health evaluation system according to claim 4,
wherein the management server enables a predeteimined administrator to edit the contents of the question items included in the physical question group, the mental question group, and the engagement question group according to the user.

12. The health evaluation system according to claim 5,
wherein the management server enables a predetermined administrator to edit the contents of the question items included in the physical question group, the mental question group, and the engagement question group according to the user.
